(19) United States
(12) Patent Application Publication
(10) Pub. No.: US 2025/0082915 A1
(43) Pub. Date: Mar. 13, 2025

(54) HEMOSTASIS VALVE USING COUNTER-ROTATING FLEXIBLE MEMBERS

(71) Applicant: Acotec Technologies Limited, San Jose, CA (US)

(72) Inventors: Thomas Kiernan, Fremont, CA (US); Vera Shinsky, Sunnyvale, CA (US)

(73) Assignee: Acotec Technologies Limited, San Jose, CA (US)

(21) Appl. No.: 18/797,449

(22) Filed: Aug. 7, 2024

Related U.S. Application Data
(60) Provisional application No. 63/581,531, filed on Sep. 8, 2023.

(51) Int. Cl. A61M 39/06

(52) U.S. Cl. CPC A61M 39/0606

(57) ABSTRACT

A hemostasis valve employs flexible members to constrict or unconstrict a collapsible gasket to close or open the valve. The hemostasis valve comprises a main housing, a rolling housing rotatable relative to the main housing, and a gasket disposed in the rolling housing. A plurality of flexible members is arranged to constrict the gasket when the rolling housing is in a first position thereby collapsing the gasket to close or narrow the lumen of the gasket, and unconstrict the gasket when the rolling housing is in a second position thereby allowing the lumen of the gasket to open.

### CROSS REFERENCE TO RELATED APPLICATIONS

This application claims priority to U.S. provisional patent application No. 63/581,531 filed Sep. 8, 2023 entitled “Large Bore Hemostasis Valve Using Counter-Rotating Flexible Members,” the disclosure of which is hereby incorporated by reference in its entirety.

### TECHNICAL FIELD

This application relates generally to medical devices. Particularly various embodiments of a hemostasis valve are described.

### BACKGROUND

Hemostasis valves are known and used in a variety of medical procedures to facilitate the passage of diagnostic or therapeutic instruments while minimizing blood loss. In a hemostasis valve, a sealing mechanism provides a secure seal around an instrument inserted.

Some conventional hemostasis valves use a flat membrane or gasket made from an elastic material. The flat gasket contains cuts or slits that can expand to accommodate different sizes of instruments. When no instrument is inserted, the slits close to prevent blood flow. However, the gasket or membrane with a geometric cut pattern tends to leak. The leaflets of the cut pattern rarely close efficiently and effectively around the instrument inserted.

Some conventional hemostasis valves use an inflatable seal that can be adjusted to fit the diameter of the inserted instrument to prevent leak, especially over the range of 0 to 9 mm. However, such hemostasis valves necessitate an inflation mechanism unwieldy to operate.

Therefore, while advancement has been made in the field of hemostasis valves, there is still a general need for improvement to overcome these and other problems of conventional hemostasis valves.

### SUMMARY

In one aspect, embodiments of the disclosure feature a hemostasis valve. In general, an embodiment of the hemostasis valve comprises a main housing, a rolling housing received in the main housing and rotatable relative to the main housing between a first position and a second position, a gasket disposed in the rolling housing, and a plurality of flexible members. The gasket comprises a proximal end secured to the proximal end of the main housing, a distal end secured to the distal end of the main housing, and a tubular body defining a lumen between the proximal end and the distal of the gasket. The tubular body is collapsible and elastic allowing the lumen to be closed and open. The plurality of the flexible members each comprises a first end coupled to the main housing, a second end coupled to the rolling housing, and extends between the rolling housing and the tubular body of the gasket. The plurality of the flexible members is arranged to constrict the tubular body of the gasket when the rolling housing is in the first position thereby collapsing the tubular body of the gasket to close or narrow the lumen, and unconstrict the tubular body of the gasket when the rolling housing is in the second position thereby allowing the lumen of the tubular body to open.

In various embodiments of the aspect, the rolling housing is rotatable relative to the main housing at an angle ranging from 30 degrees to 330 degrees.

In various embodiments of the aspect, each of the first ends of the plurality of the flexible members is coupled to the main housing in a first plane, and each of the second ends of the plurality of the flexible members is coupled to the rolling housing in a second plane proximal of the first plane. In an embodiment, the first plane and/or the second plane is (are) generally perpendicular to the central longitudinal axis of the valve.

In various embodiments of the aspect, each of the plurality of the flexible members partially circumferentially surrounds the tubular body of the gasket in extending between the rolling housing and the tubular body of the gasket.

In various embodiments of the aspect, the first ends of the plurality of the flexible members are coupled to the main housing at locations generally evenly spaced apart around a circle. Alternatively, the first ends of the plurality of the flexible members are coupled to the main housing at locations unevenly spaced apart.

In various embodiments of the aspect, the second ends of the plurality of the flexible members are coupled to the rolling housing at locations generally evenly spaced apart around a circle. Alternatively, the second ends of the plurality of the flexible members are coupled to the rolling housing at locations unevenly spaced apart.

In various embodiments of the aspect, the plurality of flexible members comprises 3-12 flexible members.

In various embodiments of the aspect, at the second position of the rolling housing, a segment of each of the plurality of the flexible members is distanced from the central longitudinal axis of the lumen of the gasket at a minimal distance. The segments of the plurality of the flexible members collectively define a passage to allow a portion of the tubular body of the gasket to pass through without constriction. At the first position of the rolling housing, the plurality of the flexible members forms an intersection that compress or constricts the portion of the tubular body of the gasket.

In various embodiments of the aspect, the gasket comprises an annular band extending outwardly from the tubular body of the gasket. The annular band can be integral of the tubular body of the gasket. In an embodiment, the annular band is located proximal of the intersection formed by the plurality of the flexible members that constricts the portion of the tubular body of the gasket.

In various embodiments of the aspect, at the second position of the rolling housing the lumen of the gasket has a diameter ranging from 4 mm to 15 mm.

In various embodiments of the aspect, the main housing comprises a side wall defining a generally cylindrical structure to receive the rolling housing therein. The side wall can be provided with an opening or openings to allow access to the rolling housing to aid a user to rotate the rolling housing relative to the main housing.

In various embodiments of the aspect, the rolling housing comprises a side wall defining a generally cylindrical structure. The cylindrical structure of the rolling housing and the cylindrical structure of the main housing can be substantially concentrically arranged.

In various embodiments of the aspect, the rolling housing further comprises one or more tab members on the side wall of the rolling housing. The one or more tab members can extend out of one or more openings in the main housing to aid the user to rotate the rolling housing relative to the main housing.

In various embodiments of the aspect, the main housing further comprises a tab member to aid the user to operate the hemostasis valve by holding the tab member of the main housing and the tab member of the rolling housing with a single hand.

In various embodiments of the aspect, the hemostasis valve further comprises a tension spring having a first end coupled to the rolling housing and a second end coupled to the main housing. The tension spring can be configured to maintain the rolling housing in the first position or provide a biasing force to return the rolling housing from the second position to the first position.

In various embodiments of the aspect, the hemostasis valve further comprises a lock mechanism configured to lock the rolling housing in the second position.

In various embodiments of the aspect, the hemostasis valve further comprises a connector configured to be coupled to the distal end of the main housing. The connector comprises a tubular body defining a passageway in communication with the lumen of the gasket and a side port for connecting with a vacuum source or flushing line.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 is a perspective view of an example hemostasis valve according to embodiments of the disclosure.

FIG. 2 is an exploded view of the hemostasis valve illustrated in FIG. 1.

FIG. 3 is another exploded view of the hemostasis valve illustrated in FIG. 1. The flexible members of the valve are omitted for clarity.

FIG. 4 is a top end view of the hemostasis valve illustrated in FIG. 1.

FIG. 5 is a cross-sectional view of the hemostasis valve illustrated in FIG. 4, taken along line A-A. The flexible members of the valve are omitted for clarity.

FIG. 6 is a cross-sectional view of the hemostasis valve illustrated in FIG. 4, taken along line B-B. The flexible members of the valve are omitted for clarity.

FIG. 7 is a cutaway side view of the hemostasis valve illustrated in FIG. 1. The hemostasis valve is shown in an open state.

FIG. 8 is another cutaway side view of the hemostasis valve illustrated in FIG. 1. The hemostasis valve is shown in a closed state.

FIG. 9 is a cutaway end view of the hemostasis valve illustrated in FIG. 1. The hemostasis valve is shown in an open state.

FIG. 10 is another cutaway end view of the hemostasis valve illustrated in FIG. 1. The hemostasis valve is shown in a closed state.

FIG. 11 is a cutaway end view of the hemostasis valve illustrated in FIG. 1. The hemostasis valve is shown in an open state. The rolling housing and the gasket of the hemostasis valve are omitted for clarity.

FIG. 12 is another cutaway end view of the hemostasis valve illustrated in FIG. 1. The hemostasis valve is shown in a closed state. The rolling housing and the gasket of the hemostasis valve are omitted for clarity.

FIG. 13 is a cutaway end view of the hemostasis valve illustrated in FIG. 1. The hemostasis valve is shown in an open state. The main housing, the rolling housing, and the gasket of the hemostasis valve are omitted for clarity.

FIG. 14 is another cutaway end view of the hemostasis valve illustrated in FIG. 1. The hemostasis valve is shown in a closed state. The main housing, the rolling housing, and the gasket of the hemostasis valve are omitted for clarity.

FIG. 15 is a cutaway side view of the hemostasis valve illustrated in FIG. 1. The hemostasis valve is shown in an open state. The main housing, the rolling housing, and the gasket of the hemostasis valve are omitted for clarity.

FIG. 16 is another cutaway side view of the hemostasis valve illustrated in FIG. 1. The hemostasis valve is shown in a closed state. The main housing, the rolling housing, and the gasket of the hemostasis valve are omitted for clarity.

FIG. 17 is a cutaway end view of the hemostasis valve illustrated in FIG. 1, showing the change of position of an end of a flexible member when the hemostasis valve operates between an open state and a closed state. The rolling housing, the gasket, and some flexible members are omitted for clarity.

FIG. 18 is a perspective view of an example gasket according to embodiments of the disclosure.

FIG. 19 is a side view of the gasket illustrated in FIG. 18.

FIG. 20 is a top end view of the gasket illustrated in FIG. 18.

FIG. 21 is a cutaway side view of the hemostasis valve illustrated in FIG. 1. The hemostasis valve is shown in an open state. The rolling housing of the hemostasis valve is omitted for clarity.

FIG. 22 is a perspective view of an example hemostasis valve comprising a lock mechanism according to embodiments of the disclosure.

### DETAILED DESCRIPTION OF EMBODIMENTS

With reference to the figures, various embodiments of a hemostasis valve will now be described. The figures are intended to facilitate description of embodiments of the disclosure and are not necessarily drawn to scale. Certain specific details may be set forth in the figures to provide a thorough understanding of the disclosure. It will be apparent to one of ordinary skill in the art that some of these specific details may not be employed to practice embodiments of the disclosure. In other instances, structures, components, systems, materials, and/or operations often associated with known medical procedures may not be shown or described in detail to avoid unnecessarily obscuring description of embodiments of the disclosure.

Embodiments of the disclosure provide a hemostasis valve. The hemostasis valve uses a plurality of flexible members strung between a stationary structure and a rolling housing to create collapsing intersections to constrict a tubular gasket running through the center of the plurality of the flexible members. The rolling housing can be rotated to allow the flexible members to loosen or unconstrict the gasket to open the valve. The unique constricting mechanism of the disclosure eliminates the need for an unwieldy inflation mechanism as used in the conventional hemostasis valves and can effectively avoid or minimize leak. The combination of a rolling housing and a plurality of flexible members provides for conformability to any cross sections of instruments to be sealed by the hemostasis valve of the disclosure.

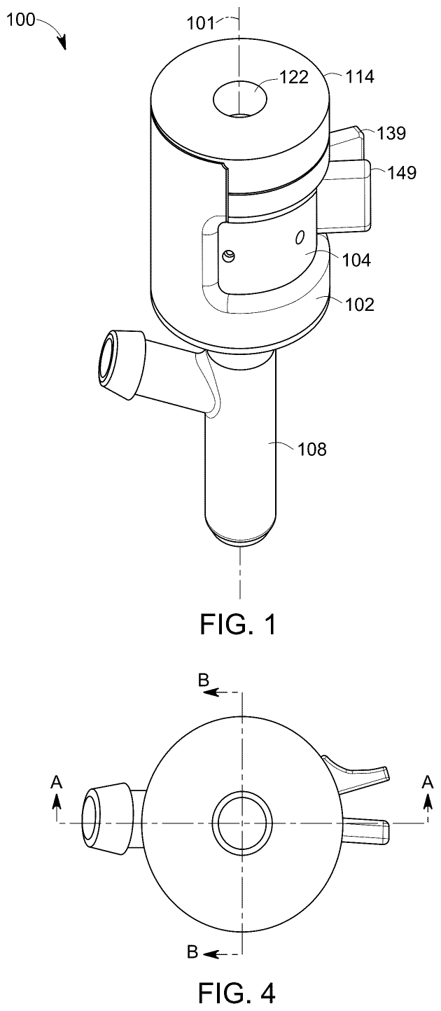

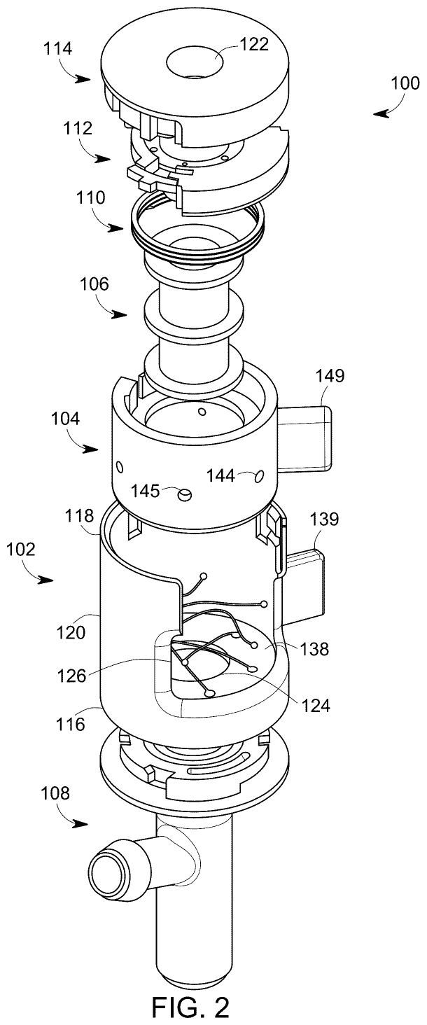

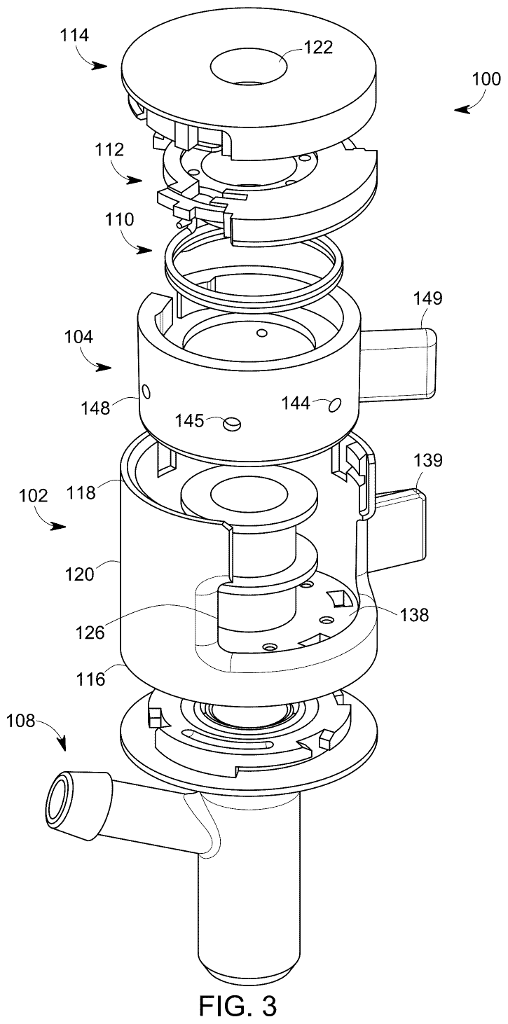

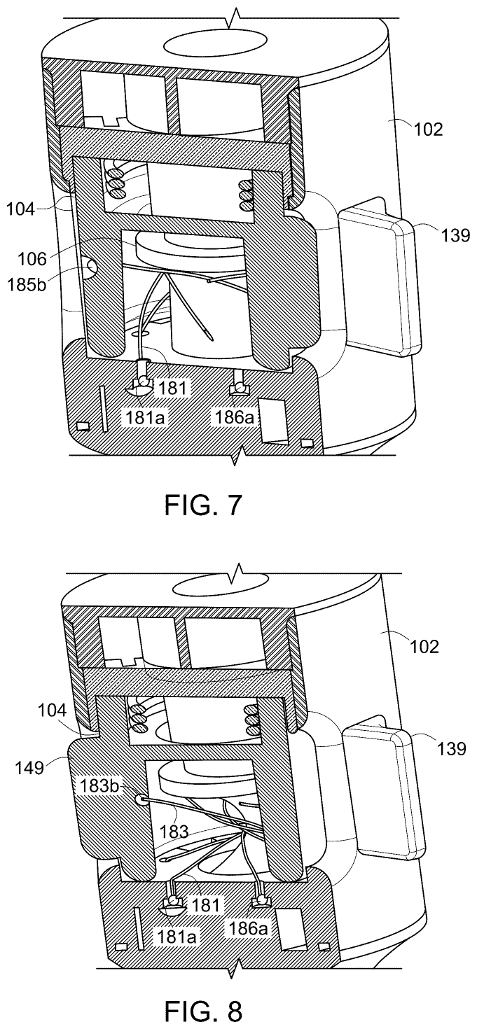

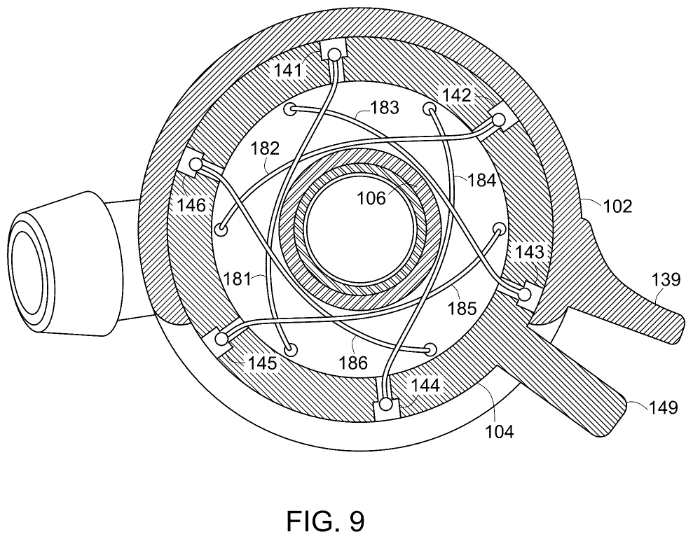

FIGS. 1-17 illustrate an example hemostasis valve 100 according to embodiments of the disclosure. In a broad overview, the hemostasis valve 100 comprises a main housing 102, a rolling housing 104, a gasket 106 (FIGS. 2-3), and a plurality of flexible members 181-186 (FIGS. 2 and 7-17). The hemostasis valve 100 may also include a connector 108, a tension spring 110, a spring housing 112, and a cap member 114 (FIGS. 2-3). As better viewed in FIGS. 9-16, each of the flexible members 181-186 includes a first end 181a-186a coupled to the main housing 102 and a second end 181b-186b coupled to the rolling housing 104. Each of the flexible members 181-186 extends in the space between the rolling housing 104 and the gasket 106 (FIGS. 7-10). The rolling housing 104 can be rotated to open or close the hemostasis valve 100. In a closed state of the hemostasis valve 100 as shown e.g., in FIGS. 8, 10, 12, 14, and 16, the plurality of the flexible members 181-186 constricts or collapses the gasket 106 thereby closing or narrowing the lumen of the gasket 106 to seal an instrument passing through the lumen of the gasket 106. In an open state of the hemostasis valve 100 as shown e.g., in FIGS. 1, 7, 9, 11, 13, and 15, the plurality of the flexible members 181-186 unconstricts or loosens the gasket 106 thereby allowing the lumen of the gasket 106 to open.

With reference to FIGS. 1-10, the main housing 102 provides a structural support and/or an assembly point of the hemostasis valve 100. The main housing 102 may provide or define a space for receiving or retaining the rolling housing 104 and includes features for securing the gasket 106. The main housing 102 includes a distal end portion 116, a proximal end portion 118, and a side wall 120 extending between the distal end portion 116 and the proximal end portion 118 (FIGS. 2-3). In an embodiment, the main housing 102 is a generally cylindrical structure providing an internal space for receiving or retaining the rolling housing 104. An opening 122 is provided in the proximal end portion 118 of the main housing 102 or in the cap member 114 for admitting an instrument (not shown) into the valve 100 (FIG. 1). An opening 124 is provided in the distal end portion 116 of the main housing 102 (FIG. 2) to allow the instrument passing through the hemostasis valve 100. The instrument inserted into and sealed by the hemostasis valve 100 of the disclosure can be any diagnostic and therapeutic instrument, including a catheter, guide wire, or any other devices having various regular or irregular cross-sectional shapes. A central axis 101 (FIG. 1) passing through the openings 122, 124 in the proximal and distal end portions of the main housing 102 defines a longitudinal axis 101 of the hemostasis valve 100.

In an embodiment, the side wall 120 of the main housing 102 can be provided with an opening 126 allowing the user to torque or rotate the rolling housing 102 in operation of the valve 100. In some embodiments, the side wall 120 of the main housing 102 can be provided with two or more openings 126 to aid the user to rotate the rolling housing 102 in operation of the valve 100. The opening 126 in the side wall 120 of the main housing 104 can be in the form of a window or slot. In an embodiment, the opening 126 in the side wall 120 of the main housing 102 may define or limit the range of angles that the rolling housing 104 can rotate relative to the main housing 102. For example, an opening window 126 can be configured to allow the rolling housing 104 to rotate about the longitudinal axis 101 of the valve 100 from about 30 degrees to about 330 degrees.

According to embodiments of the disclosure, the main housing 102 provides a plurality of locations 131-136 at which the first ends 181a-186a of the flexible members 181-186 can be coupled to the main housing 102 (FIGS. 2 and 11-12). For example, the main housing 102 may include a base member 138 provided with a plurality of apertures 131-136. Each of the apertures 131-136 can be configured to allow a flexible member to pass through, and the first end of the flexible member can be coupled or attached to the base member 138. The coupling or attachment of the first ends 181a-186a of the flexible members 181-186 to the base member 138 can be achieved by melting each of the first ends 181a-186a of the flexible members 181-186 to form a ball having an enlarged diameter, by creating a knot at each of the first ends 181a-186a of the flexible members 181-186, by bonding, by a combination thereof, or any other suitable means. In an embodiment where metallic flexible members 181-186 such as nitinol or stainless are used, the attachment or coupling to the base member 138 can be accomplished by welding the ends 181a-186a into balls, or by soldering additional sleeves into place.

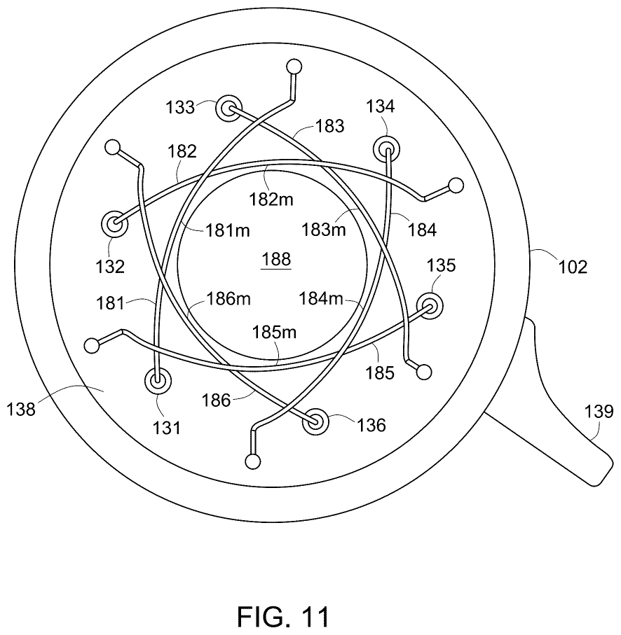

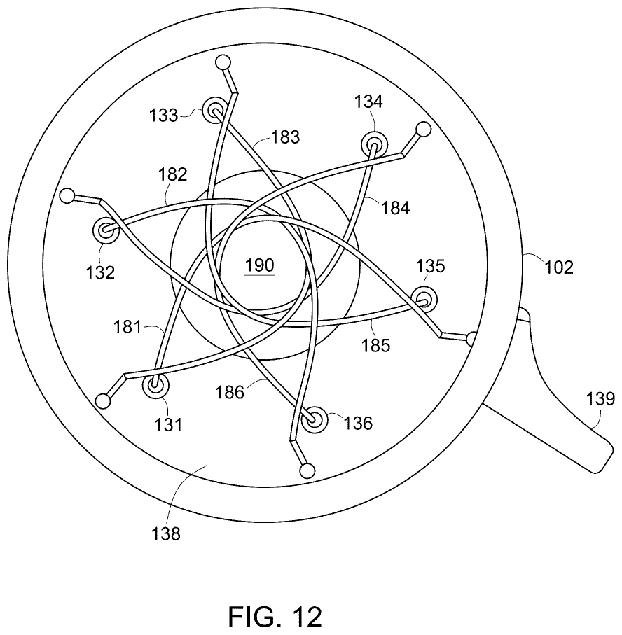

In an embodiment, the plurality of locations 131-136 at which the first ends 181a-186a of the flexible members 181-186 are coupled to the main housing 102 are generally evenly spaced apart along a circle (FIGS. 11-12). By way of example, the apertures in the base member 138 may be spaced apart in about 120 degrees if three flexible members are included in the hemostasis valve, or 90 degrees if four flexible members are used, or 60 degrees if six flexible members are used, or 40 degrees if nine flexible members are used, and so on. Alternatively, the plurality of locations 131-136 at which the first ends 181a-186a of the flexible members 181-186 are coupled to the main housing 102 can be unevenly spaced apart. In an embodiment, the plurality of locations 131-136 at which the first ends 181a-186a of the flexible members 181-186 are coupled to the main housing 102 may be in a same plane in the plate member 138.

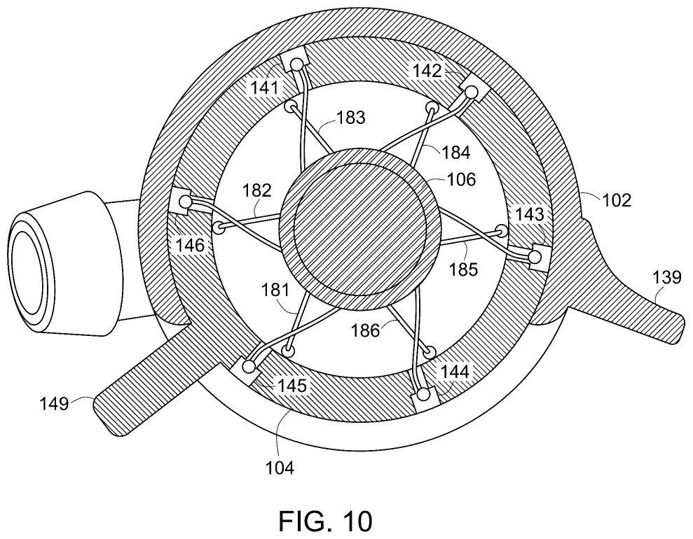

With reference to FIGS. 1-10, the rolling housing 104 can serve as an actuator to open or close the hemostasis valve 100. The rolling housing 104 is rotatable relative to the main housing 102. In an embodiment, the rolling housing is received in the main housing 102. The rolling housing 104 can be constructed or configured to receive the gasket 106 (FIGS. 2-3). The rolling housing 104 provides a plurality of locations 141-146 (FIGS. 9-10) at which the second ends 181b-186b of the flexible members 181-186 can be coupled to the rolling housing 104. When being rotated relative to the main housing 102, the rolling housing 104 changes the positions of the plurality of locations 141-146 for coupling the second ends 181b-186b of the flexible members 181-186, allowing the flexible members 181-186 to constrict or unconstrict the gasket 106 to close or open the hemostasis valve 100, to be described in greater detail below.

In an embodiment, the rolling housing 104 is generally a cylindrical structure comprising a side wall 148 defining a space for receiving the gasket 106. In an embodiment, the cylindrical structure of the rolling housing 104 is generally concentrical with the cylindrical structure of the main housing 102. In an embodiment, the side wall 148 of the rolling housing 104 is provided with a plurality of apertures 141-146 defining a plurality of locations at which the second ends 181b-186b of the flexible members 181-186 are coupled to the rolling housing 104. Each of the apertures 141-146 may be configured to allow a flexible member to pass through and allow the end of the flexible member to be coupled to the rolling housing 104 at the aperture. The coupling or attachment of the second ends 181b-186b of the flexible members 181-186 to the rolling housing at the apertures 141-146 can be achieved by melting each of the second ends 181b-186b of the flexible members 181-186 to form a ball having an enlarged diameter, by creating a knot at each of the second ends 181b-186b of the flexible members 181-186, by bonding, by a combination thereof, or any other suitable means. In an embodiment where metallic flexible member 181-186 such as nitinol or stainless are used, the attachment or coupling to the rolling housing 104 can be accomplished by welding the ends 181b-186b into balls, or by soldering additional sleeves into place.

In an embodiment, the apertures 141-146 in the side wall 148 of the rolling housing 104 are generally evenly spaced apart around a circle. By way of example, the apertures 141-146 may be spaced apart in about 120 degrees if three flexible members are included in the hemostasis valve, or 90 degrees if four flexible members are used, or 60 degrees if six flexible members are used, or 40 degrees if nine flexible members are used, and so on. Alternatively, the plurality of locations 141-146 at which the second ends 181b-186b of the flexible members 181-186 are coupled to the main housing 102 can be unevenly spaced apart. In an embodiment, the plurality of apertures 141-146 in the side wall 148 of the rolling housing 104 are in a plane. The plane can be generally perpendicular to the longitudinal axis 101 of the hemostasis valve 100.

In an embodiment, the rolling housing 104 comprises a tab member 149 to facilitate the user to operate the hemostasis valve 100. The tab member 149 may protrude outwardly from the side wall 148 of the rolling housing 104 and out of the opening 126 in the main housing 102 when assembled. The tab member 149 allows the user to apply a torque to the rolling housing 104 with ease in operating the hemostasis valve 100. In an embodiment, the main housing 102 also includes a tab member 139. For example, a tab member 139 can be provided extending outwardly from the side wall 120 adjacent to the opening 126 of the main housing 102. The tab member 139 on the main housing 102 and the tab member 149 on the rolling housing 104 allow the user to hold or grip the hemostasis valve 100 with a single hand e.g., in rotating the rolling housing 104 relative to the main housing 102 to open the valve 100, thereby allowing the user to insert an instrument into the valve 100 with another hand. The tab member 139 on the main housing 102 and the tab member 149 on the rolling housing 104 also allow the hemostasis valve 100 to be locked in an open state to aid in sterilization of the valve, to be described in more detail below. In some embodiments, the rolling housing 104 may comprise two or more tab members to facilitate the user to operate the hemostasis valve 100.

In an embodiment, the rolling housing 104 may include features supporting the tension spring 110. For example, as shown in FIGS. 2-3 the rolling housing 104 may include a circular flange protruding inwardly from the inner surface of the side wall 148 for receiving and supporting a tension spring 110. The rolling housing 104 may be provided with a slot in the side wall 148 for coupling an end of the tension spring 110. The other end of the tension spring 110 can be coupled to the proximal end portion 118 of the main housing 102, or to a spring housing 112 which is attached to the proximal end portion 118 of the main housing 102. The tension spring 110 can provide a biasing force to the rolling housing 104. In an embodiment, the tension spring 110 is configured to be in a low energy state when the rolling housing 104 is in a default position in which the valve 100 is closed. The tension spring 110 would be then in a stretched or stressed state when the rolling housing 104 is rotated relative to the main housing 102 to open the valve 100, and thus creates a biasing force to pull the rolling housing 104 back to the default closed position.

In an embodiment, the main housing 102, the rolling housing 104, and the gasket 106 are arranged or configured to allow the plurality of coupling locations 131-136 in the base member 138 of the main housing 102 to be encompassed by the rolling housing 104 or between the rolling housing 104 and the tubular gasket 106. By way of example, as shown in FIGS. 7-8, 9-10, and 11-12 an imaginary circle connecting the plurality of apertures 131-136 in the base member 138 of the main housing 102 may have a diameter smaller than the diameter of an imaginary circle connecting the plurality of apertures 141-146 in the rolling housing 104, but greater than the diameter of the tubular body 164 of the gasket 106 in the free or natural state. As such, a flexible member e.g., 181 can pass through an aperture e.g., 131 in the base member 138 of the main housing 102 with an end 181a of the flexible member 181 (the “first” end as used herein) being attached to the base member 138, extend upwardly in the space between the rolling housing 104 and the tubular gasket 106, and then pass through an aperture e.g., 141 in the rolling housing 104 with the other end 181b of the flexible member 181 (the “second” end as used herein) being attached to the rolling housing 104 (FIGS. 7-10). In an embodiment, the coupling locations 131-136 in the base member 138 of the main housing 102 are distal of the coupling locations 141-146 in the rolling housing 104. This allows a flexible member e.g., 181 to circumferentially surround or partially circumferentially surround the tubular body of the gasket 106 in extending from the first end 181a to the second end 181b of the flexible member 181, as will be described further below.

With reference to FIGS. 2-3, 5-6, and 18-20, the gasket 106 provides a secure seal around an instrument inserted into the hemostasis valve 100. The gasket 106 includes a distal end 160, a proximal end 162, and a tubular body 164 defining a lumen 166 extending between the distal end 160 and the proximal end 162. The tubular body 164 of the gasket 106 is collapsible or compliant, allowing the lumen 166 of the tubular body 164 to be closed or narrowed when the tubular body 164 is constricted. The tubular body 164 of the gasket 106 is elastic or resilient allowing it to recover or resume its free or natural state to open the lumen 166. Suitable materials for constructing the gasket 106 include silicone, urethane, ethylene-vinyl acetate, natural or synthetic rubber, or other polymers known in the art. In an embodiment, the gasket 106 is made of silicone.

The gasket 106 can be disposed in the rolling housing 104. The distal end 160 of the gasket 106 can be secured to the distal end 116 of the main housing 102. The proximal end 162 of the gasket 106 can be secured to the proximal end 118 of the main housing 102. In an embodiment, the distal end 160 of the gasket 106 comprises an outwardly extending flange 167 configured to be secured to the base member 138 of the main housing 102, and/or compressed between the base member 138 of the main housing 102 and a connector 108 (FIGS. 2-3 and 5-6). For instance, the bottom surface of the base member 138 of the main housing 102 and the upper surface of the connector 108 can include features to receive the outwardly extending flange 167 of the distal end 160, allowing it to be compressed between the base member 138 of the main housing 102 and the connector 108 and thus secured to the distal end 116 of the main housing 102 when the valve is assembled. In an embodiment, the proximal end 162 of the gasket 106 comprises an outwardly extending flange 168 configured to be compressed between the spring retaining housing 112 and the cap member 114 (FIGS. 2-3 and 5-6). For instance, the upper surface of the spring retaining housing 112 and the bottom surface of the cap member 114 can include features to receive the outwardly extending flange 168 of the proximal end 162, allowing it to be compressed between the spring retaining housing 112 and the cap member 114 and thus secured to the proximal end 118 of the main housing 104 when the valve is assembled. In an embodiment, the gasket 106 can be positioned to align the lumen 166 of the gasket 106 with the longitudinal axis 101 of the hemostasis valve 100.

In an embodiment, the tubular body 164 of the gasket 106 or the lumen 166 of the gasket 106 can be sized and shaped to receive instruments to be sealed by the hemostasis valve 100 of the disclosure. By way of example, the lumen 166 of the gasket 106 may have a diameter ranging from 4 mm to 15 mm. In an embodiment, the lumen 166 of the gasket 106 is sized for receiving an instrument having a maximal cross-sectional dimension ranging from 0.1 mm to 14.5 mm. It should be noted that the above dimensions are provided for illustration and understanding of embodiments of the disclosure. One of ordinary skill in the art can readily scale the size of lumen 166 of the gasket 106 based on specific applications, and the appended claims of the disclosure are not limited to the specific dimensions.

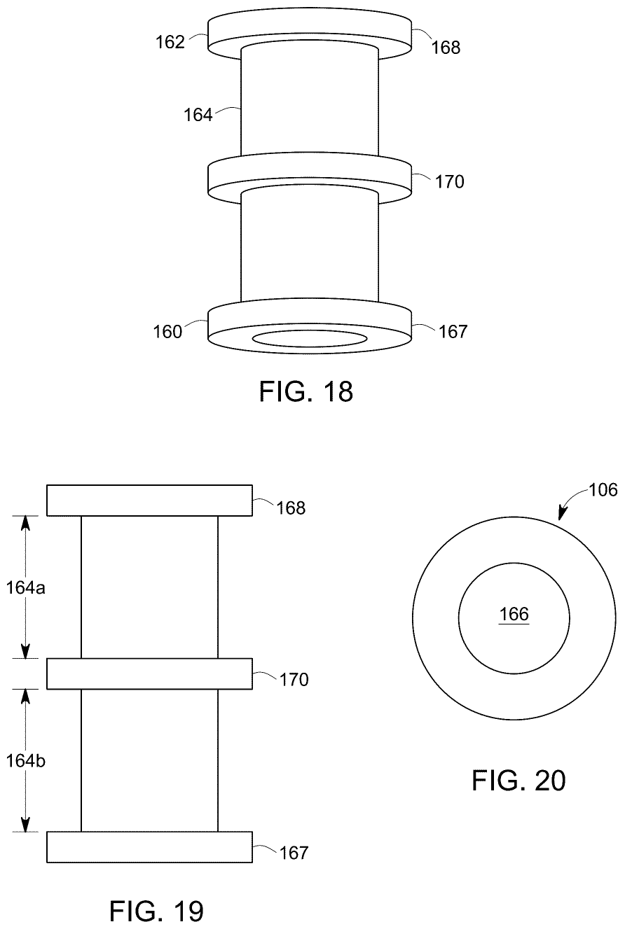

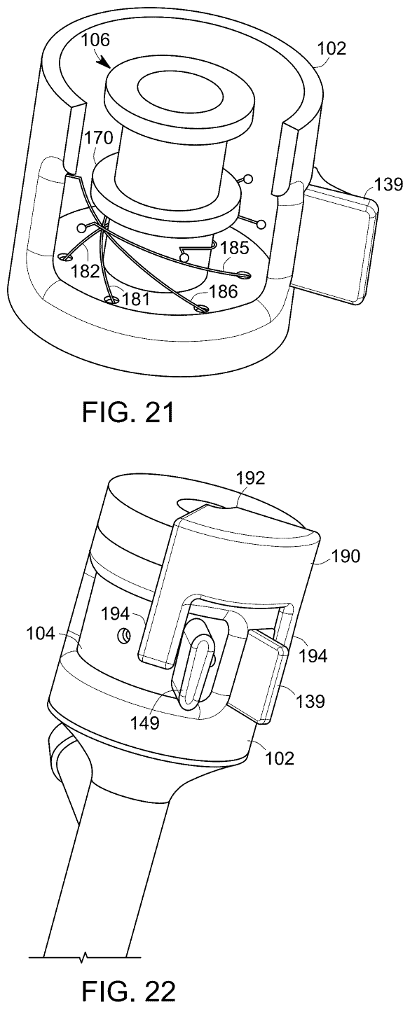

With reference to FIGS. 18-21, an example gasket 106 according to embodiments of the disclosure comprises an annular band 170 extending outwardly from the outer surface of the tubular body 164 of the gasket 106. The annular band 170 can prevent the tubular body 164 from folding and/or twisting when the gasket 106 is constricted by the flexible members 181-186 to close the hemostasis valve 100. As illustrated in FIG. 21, the annular band 170 can be located above the level of locations at which the second ends 181b-186b of the flexible members 181-186 are coupled to the rolling housing 104. This arrangement would allow the plurality of the flexible members 181-186 to constrict or tighten the tubular body 164 of the gasket at a location(s) below the annular band 170 when the hemostasis valve 100 is in a closed state. The annular band 170 can prevent the portion 164a of the tubular body 164 above the annular band 170 from folding over when the portion 164b of the tubular body 164 below the annular band 170 is constricted. In an embodiment, the annular band 170 is an integral portion of the tubular body 164 of the gasket 170 and constructed with the same material of the gasket 106. Alternatively, the annular band 170 is a separate part bonded to the tubular body 164 of the gasket 106 via any suitable means. According to alternative embodiments of the disclosure, the gasket 106 may include a plurality of rib members disposed on the outer surface of the tubular body to prevent folding of the tubular body of the gasket in operation of the valve.

With reference to FIGS. 7-16, the flexible members 181-186 are constructed, arranged, or configured to constrict the tubular body 164 of the gasket 106 to close or narrow the lumen 166 of the tubular body 164. The flexible members 181-186 are further constructed, arranged, or configured to unconstrict the tubular body 164 of the gasket 106 to allow the tubular body 164 of the gasket 106 to return to its free or natural state or to allow the gasket 106 to open the lumen 166.

The flexible members 181-186 can be in the form of filaments, strings, flat wires, ribbons, or the like. A flexible member (e.g., any of 181-186) can comprise a single strand e.g., a mono-filament. In some embodiments, a flexible member can comprise multiple strands that are grouped, twisted, or woven to form the flexible member. Suitable materials for constructing the flexible members 181-186 include polymers and/or metals. By way of examples, the flexible members 181-186 can be made of polypropylenes, nylon, nitinol, stainless steel, or the like. For illustration, six flexible members 181-186 are shown in FIGS. 7-16. It should be noted that fewer or more than six flexible members e.g., 3-12 can be included in the hemostasis valve 100 of the disclosure.

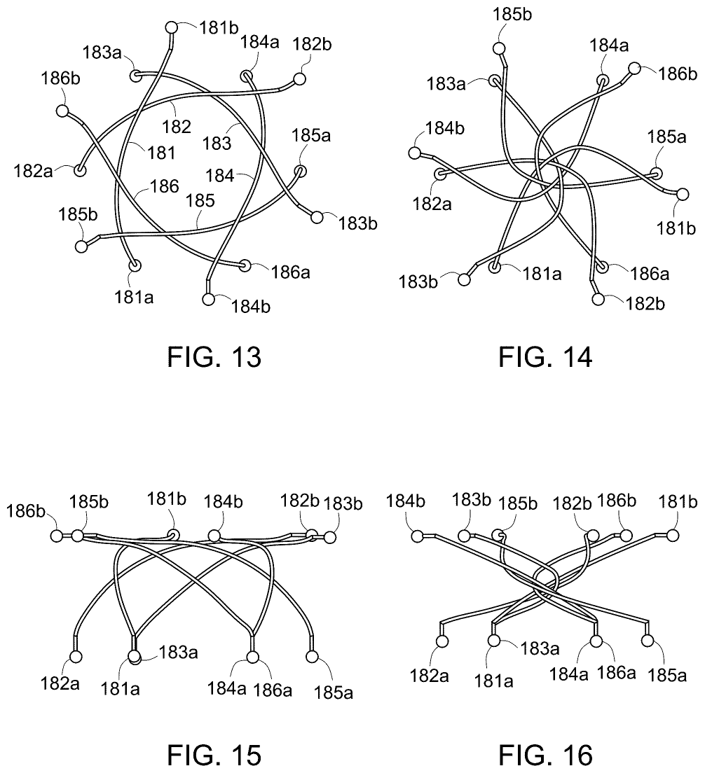

Each of the flexible members 181-186 comprises a first end 181a-186a coupled to the main housing 102 and a second end 181b-186b coupled to the rolling housing 104 (FIGS. 13-16). In an embodiment, the flexible members 181-186 extend from their first ends 181a-186a to their second ends 181b-186b respectively, in the space between the rolling housing 104 and the tubular body 164 of the gasket 106 (FIGS. 9-10).

In an embodiment, the first ends 181a-186a of the flexible members 181-186 are coupled to the main housing 102 at locations distal to the locations at which the second ends 181b-186b of the flexible members 181-186 are coupled to the rolling housing 104 (FIGS. 15-16). In an embodiment, the first ends 181a-186a of the flexible members 181-186 are coupled to the main housing 102 at locations that are generally in a same plane (FIGS. 15-16) e.g., in the base member 138 of the main housing 102. In an embodiment, the second ends 181b-186b of the flexible members 181-186 are coupled to the rolling housing 104 at locations that are generally in a same plane (FIGS. 15-16.

In an embodiment, the first ends 181a-186a of the flexible members 181-186 are coupled to the main housing 102 at locations 131-136 that are generally evenly spaced apart around a circle (FIGS. 11-12). By way of example, the locations 131-136 in the main housing 102 (FIGS. 11-12) for coupling the first ends 181a-186a of the flexible members 181-186 may be spaced apart in about 120 degrees if three flexible members are included in the hemostasis valve, or 90 degrees if four flexible members are used, or 60 degrees if six flexible members are used, or 40 degrees if nine flexible members are used, and so on. Likewise, the second ends 181b-186b of the flexible members 181-186 can be coupled to the rolling housing 104 at locations 141-146 that are generally evenly spaced apart around a circle (FIGS. 9-10). By way of example, the locations in the rolling housing 104 for coupling the second ends 181b-186b of the flexible members 181-186 can be spaced apart in about 120 degrees if three flexible members are included in the hemostasis valve, or 90 degrees if four flexible members are used, or 60 degrees if six flexible members are used, or 40 degrees if nine flexible members are used, and so on. Alternatively, the first ends 181a-186a of the flexible members 181-186 are coupled to the main housing 102 at locations 131-136 that are unevenly spaced apart. Similarly, the second ends 181b-186b of the flexible members 181-186 can be coupled to the rolling housing 104 at locations 141-146 unevenly spaced apart.

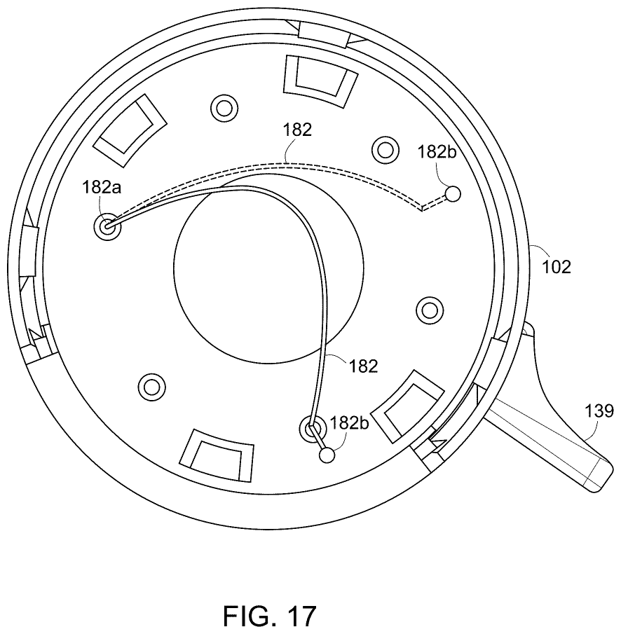

The positions of the second or proximal ends 181b-186b of the flexible members 181-186 change as the rolling housing 104 is rotated between e.g., a default first position in which the valve 100 is closed and a second position in which the valve 100 is open. For illustration, FIG. 17 depicts the change of position of the second end 182b of flexible member 182. In the second position of the rolling housing 104 in which the valve 100 is open, the flexible member 182 (shown in phantom lines) extends from the distal first end 182a to the proximal second end 182b in the space between the rolling housing 104 and the gasket 106, partially circumferentially surrounding the tubular body 164 of the gasket 106. The length of the flexible member 182 can be selected so that while the flexible member 182 partially circumferentially surrounds the tubular body 164 of the gasket 106, it does not compress or constrict the tubular body 164 of the gasket 106, allowing the tubular body 164 of the gasket 106 to be in a natural state or allowing the gasket 106 to open the lumen. As the rolling housing 104 rotates e.g., to the default first position, the position of the second end 182b changes. In the default first position of the rolling housing 104, the flexible member 182 (shown in a solid line) is stretched and compresses the tubular body 164 of the gasket 106. Collectively, the plurality of the flexible members 181-186 form an intersection that constricts, collapses, or tightens the tubular body 164 of the gasket 106 to close or narrow the lumen 166 of the gasket 106. Accordingly, with the rolling housing 104 rotating between a first position and a second position, the flexible members 181-186 are “rotated” or “counter-rotated” to close or open the hemostasis valve 100. According to an embodiment of the disclosure, the hemostasis valve 100 comprises a tension spring 110 coupled to the rolling housing 104 and the main housing 102. The tension spring 110 can be constructed or configured to be in a low-energy or natural state when the rolling housing 104 is in the default first position in which the flexible members 181-186 form an intersection(s) to constrict or tighten the tubular body 164 of the gasket 106. The tension spring 110 would be in a stretched or stressed state when the rolling housing 104 is in a second position in which the flexible members 181-186 unconstrict the tubular body 164 of the gasket 106 to open the valve 100. The tension spring 110 in the stressed state generates a tension or biasing force to pull or bring the rolling housing 104 from the second position to the default first position.

With reference to FIGS. 9-16, example arrangements or configurations of the flexible members 181-186 according to embodiments of the disclosure are shown. FIG. 9 is a cutaway end view showing a configuration of the flexible members 181-186 when the hemostasis valve 100 is in an open state. FIG. 10 is a cutaway end view showing a configuration of the flexible members 181-186 when the hemostasis valve 100 is in a closed state. In FIGS. 11-12, the rolling housing 104 and the gasket 106 of the hemostasis valve 100 are omitted for clarity. In FIGS. 13-16, only the flexible members 181-186 in the open and close configurations are shown whereas the main housing 102, the rolling housing 104, and the gasket 106 of the hemostasis valve 100 are omitted for clarity.

As shown in FIGS. 9, 11, 13, and 15, in an open state of the hemostasis valve 100 each of the flexible members 181-186 has a point or segment 181m-186m (FIG. 11) distanced from the tubular body 164 of the gasket 106 or distanced from the central longitudinal axis of the lumen 166 of the gasket 106 at a minimum distance. The length of the flexible members 181-186 is chosen so that the points or segments 181m-186m of the flexible members 181-186 do not constrict the tubular body 164 of the gasket 106, thereby allowing the tubular body 164 of the gasket 106 to be in a free or natural state. Collectively, the segments 181m-186m of the flexible members 181-186 define a central passage 188 (FIG. 11) having a minimal size allowing the tubular body 164 or a portion of the tubular body 164 to pass without any constriction. In an embodiment, the segments 181m-186m of the flexible members 181-186 are generally in a plane perpendicular to the longitudinal axis 101 of the valve 100.

As shown in FIGS. 10, 12, 14, 16, in a closed state of the hemostasis valve 100 the second ends 181b-186b of the flexible members 181-186 change positions, allowing the flexible members 181-186 to stretch and compress the tubular body 164 of the gasket 106. Collectively, the plurality of flexible members 181-186 form an intersection 190 with a reduced size constricting or tightening the tubular body 164 of the gasket 106.

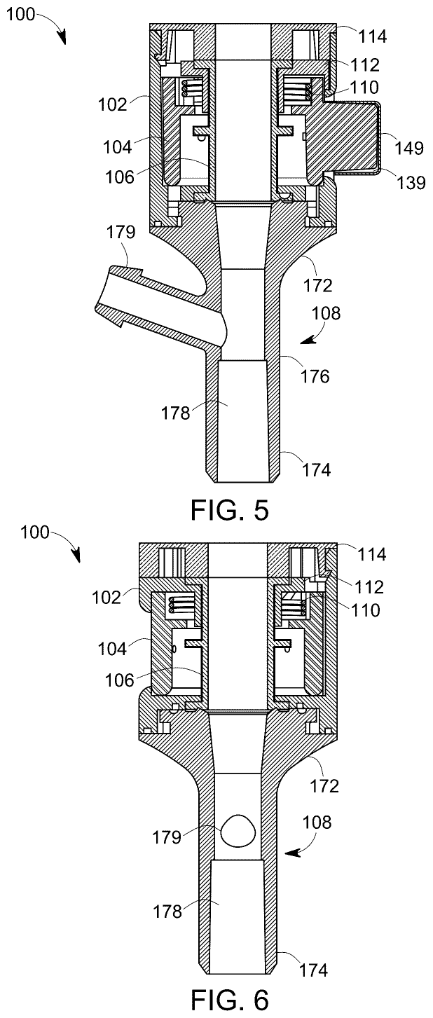

With reference to FIGS. 1-3 and 5-6, an embodiment the hemostasis valve 100 of the disclosure comprises a connector 108. The connector 108 comprises a proximal end 172, a distal end 174, and tubular body 176 defining a passageway 178 between the proximal end 172 and the distal end 174 (FIGS. 5-6). The proximal end 172 of the connector 108 includes features such as snap-fit features for attachment to the distal end 116 of the main housing 102 of the hemostasis valve 100. The proximal end 172 of the connector 108 may also include features such as a recess or groove for receiving and/or securing the distal end 160 of the gasket 106 to the main housing 102. When assembled, the passageway 178 of the connector 108 is in communication with the lumen 166 of the gasket 106. The passageway 178 of the connector 108 can be sized and shaped to receive an instrument to be used in connection with the hemostasis valve 100. In an embodiment, the connector 108 includes one or more side ports 179 in communication with the passageway 178 of the connector 108. For example, the connector 108 may include a side port 179 for coupling to a vacuum source useful in an aspiration medical procedure or to a flush line to minimize clot formation.

With reference now to FIG. 22, in an embodiment the hemostasis valve 100 of the disclosure comprises a lock 190 configured to keep the valve 100 in the open state. The lock 190 may include a restraining element such as a clip feature 192 for attachment to the valve 100. The lock 190 may include spaced apart leg members 194 configured to constrain the tab member 149 of the rolling housing 104 with the tab member 139 of the main housing 102 in the open state of the valve 100, thereby preventing the rolling housing 104 from returning to its default closed position by the biasing force of the tension spring 110. The locked open state of the valve 100 allows the lumen 166 of the gasket 106 to remain open, aiding in sterilization of the tubular body 164 of the gasket 106 in its natural state or of the other parts of the valve.

Various embodiments of a hemostasis valve have been described with reference to figures. It should be noted that an aspect described in conjunction with a particular embodiment is not necessarily limited to that embodiment and can be practiced in any other embodiments. The figures are intended for illustration of embodiments but not for exhaustive description or limitation on the scope of the disclosure. Alternative structures, components, and materials will be readily recognized as being viable without departing from the principle of the claimed invention. For instance, while various embodiments of a constricting mechanism are described in conjunction with figures showing six flexible members, the constricting mechanism can include a different number of flexible members such as three, four, five, seven, eight, nine, ten, eleven, twelve, and so on. The length of the flexible members and/or the rotation degrees of the rolling housing can be configured to effectively constrict or unconstrict the tubular body of the gasket using a different number of flexible members. While a fewer number of flexible members may cause less concentric constriction of the tubular body of the gasket or poorer sealing effect, the less desirable effect can be compensated by longer flexible members and a greater degree of rotation of the rolling housing. For example, a rotation of the rolling housing in 250 degrees or more would allow longer flexible members to loop or wrap around the tubular body of the gasket, ameliorating the less concentric constriction effect caused by e.g., three flexible members rotating only in 45 degrees. The inconvenience of needing to rotate the rolling housing in greater degrees can be overcome with gearing mechanisms. On the other hand, while use of a greater number of flexible members (e.g., nine) may complicate the assembly of the hemostasis valve, it can provide the benefit of more concentric constriction of the gasket with shorter flexible members and lesser degree of rotation of the rolling housing (e.g., 45 degrees). In general, the length of the flexible members should be chosen such that it would not limit the flexible members' ability to rotate through the center of the device or the ability to compress the gasket. One of ordinary skill in the art can readily optimize a hemostasis valve design based on application specifications by properly choosing the number and length of the flexible members and rotation degrees of the rolling housing to maximize hemostasis and balance ease of assembly and human use element. Therefore, the disclosure and appended claims are not limited to the specific number and length of the flexible members, and the rotation degrees of the rolling housing.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. Relative terms such as “above,” “below,” “upper,” “under,” “top,” “bottom,” or the like are used for ease of description of various embodiments, but do not necessarily represent an orientation used during manufacturing or use. The term “proximal” and its grammatically equivalent refers to a position, direction or orientation towards the user or physician's side. The term “distal” and its grammatically equivalent refers to a position, direction, or orientation away from the user or physician's side. The terms “first” and “second” etc. are used to distinguish one element from another in describing various similar elements. The terms “first” and “second” may include references to two or more than two. Further, the use of the terms “first” and “second” should not be construed as in a particular order unless the context clearly dictates otherwise. All numeric values are provided for illustration and assumed to be modified by the term “about,” whether explicitly indicated or not. The term “about” generally refers to a range of numbers that one of skill in the art would consider equivalent to the recited value e.g., having the same function or result. The term “about” may include numbers that are rounded to the nearest significant figure. The recitation of a numerical range by endpoints includes all numbers within that range.

Those skilled in the art will appreciate that various other modifications may be made. All these or other variations and modifications are contemplated by the inventors and within the scope of the invention.

What is claimed is:

1. A hemostasis valve, comprising:
a main housing having a proximal end and a distal end;
a rolling housing rotatable relative to the main housing between a first position and a second position;
a gasket disposed in the rolling housing, the gasket comprising a proximal end secured to the proximal end of the main housing, a distal end secured to the distal end of the main housing, and a tubular body defining a lumen between the proximal end and the distal of the gasket, the tubular body being collapsible and elastic allowing the lumen to be closed and open; and
a plurality of flexible members, each of the plurality of the flexible members comprising a first end coupled to the main housing, a second end coupled to the rolling housing, and extending between the rolling housing and the tubular body of the gasket, the plurality of the flexible members being arranged to constrict the tubular body of the gasket when the rolling housing is in the first position thereby collapsing the tubular body of the gasket to close or narrow the lumen, and unconstrict the tubular body of the gasket when the rolling housing is in the second position thereby allowing the lumen of the tubular body to open.

2. The hemostasis valve of claim 1, wherein the rolling housing is rotatable relative to the main housing at an angle ranging from 30 degrees to 330 degrees.

3. The hemostasis valve of claim 1, wherein each of the first ends of the plurality of the flexible members is coupled to the main housing in a first plane, and each of the second ends of the plurality of the flexible members is coupled to the rolling housing in a second plane proximal of the first plane.

4. The hemostasis valve of claim 3, wherein each of the plurality of the flexible members partially circumferentially surrounds the tubular body of the gasket in extending between the rolling housing and the tubular body of the gasket.

5. The hemostasis valve of claim 3, wherein the first ends of the plurality of the flexible members are coupled to the main housing at locations generally evenly spaced apart around a circle.

6. The hemostasis valve of claim 3, wherein the second ends of the plurality of the flexible members are coupled to the rolling housing at locations generally evenly spaced apart around a circle.

7. The hemostasis valve of claim 3, wherein the first ends of the plurality of the flexible members are coupled to the main housing at locations unevenly spaced apart, and/or the second ends of the plurality of the flexible members are coupled to the rolling housing at locations unevenly spaced apart.

8. The hemostasis valve of claim 3, wherein the plurality of flexible members comprises 3-12 flexible members.

9. The hemostasis valve of claim 3, wherein at the second position of the rolling housing, a segment of each of the plurality of the flexible members is distanced from the central longitudinal axis of the lumen of the gasket at a minimal distance, the segments of the plurality of the flexible members collectively define a passage to allow a portion of the tubular body of the gasket to pass through without constriction.

10. The hemostasis valve of claim 9, wherein at the first position of the rolling housing, the plurality of the flexible members forms an intersection that compress or constricts the portion of the tubular body of the gasket.

11. The hemostasis valve of claim 10, wherein the gasket comprises an annular band outwardly extending from the tubular body of the gasket, the annular band being located proximal of the intersection.

12. The hemostasis valve of claim 1, wherein the gasket comprises an annular band extending outwardly from the tubular body of the gasket.

13. The hemostasis valve of claim 12, wherein the annular band is integral of the tubular body of the gasket.

14. The hemostasis valve of claim 1, wherein at the second position of the rolling housing the lumen of the gasket has a diameter ranging from 4 mm to 15 mm.

15. The hemostasis valve of claim 1, wherein the main housing comprises a side wall defining a generally cylindrical structure to receive the rolling housing therein, the side wall being provided with one or more openings to allow access to the rolling housing to aid a user to rotate the rolling housing relative to the main housing.

16. The hemostasis valve of claim 15, wherein the rolling housing comprises a side wall defining a generally cylindrical structure, the cylindrical structure of the rolling housing and the cylindrical structure of the main housing being substantially concentrically arranged.

17. The hemostasis valve of claim 16, wherein the rolling housing further comprises one or more tab members on the side wall of the rolling housing, the one or more tab members extending out of the one or more openings in the main housing respectively to aid the user to rotate the rolling housing relative to the main housing.

18. The hemostasis valve of claim 17, wherein the main housing further comprises a tab member to aid the user to operate the hemostasis valve by holding the tab member of the main housing and the one or more tab members of the rolling housing with a single hand.

19. The hemostasis valve of claim 1, further comprising a tension spring having a first end coupled to the rolling housing and a second end coupled to the main housing, the tension spring being configured to maintain the rolling housing in the first position or provide a biasing force to return the rolling housing from the second position to the first position.

20. The hemostasis valve of claim 19, further comprising a lock mechanism configured to lock the rolling housing in the second position.

21. The hemostasis valve of claim 1, further comprising a connector configured to be coupled to the distal end of the main housing, the connector comprising a tubular body defining a passageway in communication with the lumen of the gasket and a side port for connecting with a vacuum source or flushing source.
